(19) United States
(12) Patent Application Publication
(10) Pub. No.: US 2007/0066930 A1
(43) Pub. Date: Mar. 22, 2007

(54) Iontophoresis device and method of producing the same

(75) Inventors: Akihiko Tanioka, Ohota-ku (JP); Akihiko Matsumura, Shibuya-ku (JP); Takehiko Matsumura, Shibuya-ku (JP); Mizuo Nakayama, Shibuya-ku (JP); Hidero Akiyama, Shibuya-ku (JP)

Correspondence Address: SEED INTELLECTUAL PROPERTY LAW GROUP PLLC, 701 FIFTH AVE, SUITE 5400, SEATTLE, WA, 98104, US

(73) Assignee: Transcutaneous Technologies, Inc., Shibuya-ku (JP)

(21) Appl. No.: 11/471,973

(22) Filed: Jun. 20, 2006

(30) Foreign Application Priority Data
Sep. 20, 2005 (JP) 2005-179953

(51) Int. Cl. A61N 1/30; A61L 33/00; B32B 18/00

(52) U.S. Cl. 604/20; 427/2.1; 264/134

(57) ABSTRACT

An iontophoresis device and method of producing the same may reduce material loss during the course of production of a conventional iontophoresis device, and may allow for easy automation of production processes and increases in production scale. The iontophoresis device may be used for administering drug ions of a first polarity generated by dissociation of a drug to a living body, and may comprise: a first conductive layer formed on a surface of a first substrate; a drug layer made of a drug coating containing the drug, the drug layer being laminated on the first conductive layer; and a first ion exchange layer made of an ion exchange coating containing an ion exchange resin having an exchange group introduced thereto, the ion exchange group having a counter ion to the first polarity ions, the first ion exchange layer being laminated on the drug layer.

### BACKGROUND OF THE INVENTION

1. Field of the Invention

The present disclosure relates to an iontophoresis device for administering drug ions of a first polarity to a living body and a method of producing the same.

2. Description of the Related Art

An iontophoresis device generally includes an active electrode structure that holds a drug whose active ingredient dissociates into positive or negative drug ions, and a counter electrode structure that functions as a counter electrode to the active electrode structure. A voltage or electrical potential having the same polarity as that of the drug ions is applied to the active electrode structure under the condition that both structures maintain ion transferring engagement with the skin (or mucosa) of a living body (a human being or an animal), whereby the drug ions are administered to the living body.

Current or electrical potential supplied to the active electrode structure causes the movement of the drug ions to the living body and the release of biological counter ions (i.e., ions that are present in the living body that have a polarity opposite to that of the drug ions) toward the active electrode structure. Biological counter ions (e.g., Na+, Cl−) each having a high mobility due to its small molecular weight are mainly released from the living body. The transport number (ratio of current contributing to the movement of the drug ions to the entire current supplied to the active electrode structure), which is the administration efficiency of the drug ions, thus decreases.

FIGS. 10A and 10B respectively show a cross sectional view and a bottom view of an iontophoresis device 101a that addresses the above mentioned problem. The iontophoresis device 101a is described in JP 3030517 B, which is incorporated herein by reference in its entirety. FIGS. 11A and 11B respectively show a cross sectional view and a bottom view of an iontophoresis device 101b that addresses the above mentioned problem. The iontophoresis device 101b is described in JP 2000-229128 A, which is incorporated herein by reference in its entirety.

The iontophoresis device 101a includes: an active electrode structure 110a comprising of a container 111, a first electrode 112 housed in the container 111, a drug holding portion 115 that holds a drug solution whose active ingredient dissociates into drug ions of a first polarity, and a first ion exchange membrane 116 that selectively passes ions of the first polarity; and a counter electrode structure 120a having a second electrode 122; and a power source 130.

When current is supplied under the condition that the active electrode structure 110a and the counter electrode structure 120a are kept in ion translating relation with a skin 40 of a living body, biological counter ions may not pass through the first ion exchange membrane 116, while the drug ions in the drug holding portion 115 are administered to the living body through the first ion exchange membrane 116. Consequently, there is a functional effect that the current used to move the biological counter ions to the drug holding portion 115 is reduced, thus enhancing the drug administration efficiency.

However, in order to produce the above mentioned iontophoresis device 101a, it is necessary to prepare the ion exchange membrane 116 in accordance with the size and shape of the active electrode structure 110a by cutting or punching, with the result that a cutting margin or a punching margin is wasted.

Furthermore, in assembling the active electrode structure 110a, it is necessary to handle a member in a wet state (the drug holding portion 115). Furthermore, it is necessary to configure the drug holding portion 115 in a fluid-tight state so that a liquid path through which biological counter ions can move between the drug holding portion 115 and the skin 40 without that passes through the first ion exchange membrane 116 is not formed. Accordingly, skill or experience is required to some degree for assembling the active electrode structure 110a. It may thus be difficult to reduce production costs, difficult to automate and difficult to mass produce.

For the iontophoresis device 101b shown in FIGS. 11A and 11B, a first electrode 112, a first electrolyte holding portion 113 that holds an electrolyte solution, a second ion exchange membrane 114 that selectively passes ions of a second polarity, a drug holding portion 115 that holds a drug solution, and a first ion exchange membrane 116 that selectively passes the ions of the first polarity are placed in a container 111 of an active electrode structure 110b. A second electrode 122, a second electrolyte holding portion 123 that holds an electrolyte solution, a third ion exchange membrane 124 that selectively passes the ions of the first polarity, a third electrolyte holding portion 125 that holds an electrolyte solution, and a fourth ion exchange membrane 126 that selectively passes the ions of the second polarity are placed in a container 121 of a counter electrode structure 120.

Movement of biological counter ions to the drug holding portion 115 is also interrupted by the first ion exchange membrane 116 with the iontophoresis device 101b. Therefore, a functional effect similar to that of the iontophoresis device 101a may be achieved, and in addition, movement of the drug ions to the first electrolyte holding portion 113 may be interrupted by the second ion exchange membrane 114. Consequently, the drug ions may be prevented from decomposing in the vicinity of the first electrode 112. Furthermore, H+ ions and OH− ions generated by an electrolytic reaction at the first electrode 112 and the second electrode 122 may be prevented from moving to the drug holding portion 115 and the third electrolyte holding portion 125 by the second ion exchange membrane 114 and the third ion exchange membrane 124, respectively. Consequently, the additional functional effect of suppressing variations in pH at the interface between the drug holding portion 115 and the skin, and at the interface between the third electrolyte holding portion 125 and the skin may be obtained.

However, the iontophoresis device 101b has problems similar to those described with respect to the iontophoresis device 101a. In addition, the number of members required to be handled in a wet state increases. Furthermore, it is necessary to keep the interface between the first electrolyte holding portion 113 and the drug holding portion 115, and the interface between the second electrolyte holding portion 123 and the third electrolyte holding portion 125, in a fluid tight state. For those reasons, it becomes more difficult to achieve the reduction in a production cost, automation of production, or mass production.

### BRIEF SUMMARY OF THE INVENTION

In view of the problems described above, in at least one embodiment an iontophoresis device and a method of producing the same may be capable of reducing material loss during the course of production.

In at least one embodiment, an iontophoresis device and a method of producing the same may be capable of simplifying a production process.

In at least one embodiment, an iontophoresis device and a method of producing the same may be capable of enhancing production yield.

In at least one embodiment, an iontophoresis device and a method of producing the same may be capable of automating production or enlarging the scale of production.

In at least one embodiment, an iontophoresis device and a method of producing the same may have a reduced production cost.

In at least one embodiment, an iontophoresis device for administering drug ions of a first polarity, generated by dissociation of a drug, to a living body, may comprise an active electrode structure comprising:

a first conductive layer formed on a surface of a first substrate;

a drug layer comprising a drug coating containing the drug, the drug layer being laminated on the first conductive layer; and

a first ion exchange layer comprising an ion exchange coating containing an ion exchange resin containing an exchange group having a counter ion to the first polarity ions, the first ion exchange layer being laminated on the drug layer.

In at least one embodiment, a method of producing an iontophoresis device for administering drug ions of a first polarity generated by dissociation of a drug to a living body, may comprise:

forming a first conductive layer on a surface of a first substrate;

forming a drug layer by applying a drug coating containing the drug to the first conductive layer; and

forming a first ion exchange layer by applying an ion exchange coating containing an ion exchange resin containing an exchange group having a counter ion to the first polarity ions to the drug layer.

In at least one embodiment, the drug layer and the first ion exchange membrane are each formed by applying a coating in order to reduce waste due to punching or cutting. It may be possible to easily automate production processes and increase production scale.

In at least one embodiment, the active electrode structure may further comprise:

a first electrolyte layer comprising an electrolyte coating containing an electrolyte, the first electrolyte layer being laminated on the first conductive layer; and

a second ion exchange layer comprising an ion exchange coating containing an ion exchange resin containing an exchange group having a counter ion to a second polarity ion, the second ion exchange layer being laminated on the first electrolyte layer,

wherein the drug layer is laminated on the second ion exchange layer.

In at least one embodiment, the method may further comprise:

forming a first electrolyte layer by applying an electrolyte coating containing an electrolyte to the first conductive layer; and

forming a second ion exchange layer by applying an ion exchange coating containing an ion exchange resin containing an exchange group having a counter ion to second polarity ions to the first electrolyte layer,

wherein the drug layer is formed on the second ion exchange layer.

Accordingly, while basic functional effects such as reduction in waste, automated production processes, and increases in production scale may be achieved, additional effects such as a reduction in drug ions decomposition in the vicinity of the first conductive layer and a reduction in movement of H+ ions or OH− ions generated at the first conductive layer to the drug layer may also be achieved to further enhance biocompatibility and stability.

In at least one embodiment, the iontophoresis device may further comprise a counter electrode structure comprising:

a second conductive layer formed on a surface of a second substrate;

a second electrolyte layer comprising an electrolyte coating containing an electrolyte, the second electrolyte layer being laminated on the second conductive layer;

a third ion exchange layer comprising an ion exchange coating containing an ion exchange resin containing an exchange group having a counter ion to the first polarity ions, the third ion exchange layer being laminated on the second electrolyte layer;

a third electrolyte layer comprising an electrolyte coating containing an electrolyte, the third electrolyte layer being laminated on the third ion exchange layer; and

a fourth ion exchange layer comprising an ion exchange coating containing an ion exchange resin containing an exchange group having a counter ion to the second polarity ions, the fourth ion exchange layer being laminated on the third electrolyte layer.

In at least one embodiment, the method may further include:

forming a second conductive layer to a surface of a second substrate;

forming a second electrolyte layer by applying an electrolyte coating containing an electrolyte to the second conductive layer;

forming a third ion exchange layer by applying an ion exchange coating containing an ion exchange resin containing an exchange group having a counter ion to the first polarity ions to the second electrolyte layer;

forming a third electrolyte layer by applying an electrolyte coating containing an electrolyte to the third ion exchange layer; and

forming a fourth ion exchange layer by applying an ion exchange coating containing an ion exchange resin containing an exchange group having a counter ion to the second polarity ions to the third electrolyte layer.

Accordingly, an additional effect such as a reduction in the movement of H+ ions or OH− ions generated at the second conductive layer to the third electrolyte layer may be achieved to further enhance biocompatibility and stability.

In at least one embodiment, the drug coating and/or the electrolyte coating may further include a water-soluble polymer. This may enhance the coating properties or film formation properties of the coatings.

In at least one embodiment, the first to fourth ion exchange layers may be non-water-soluble coating films. This may serve to make the quality of the iontophoresis device more uniform.

In at least one embodiment, the ion exchange coating may contain a low molecular weight polyethylene, ultra-high molecular weight polyvinyl alcohol (PVA), or chitosan, or a mixture thereof. This may provide non-water-soluble properties to the first to fourth ion exchange layers through a simple treatment, while enhancing safety.

In at least one embodiment the first electrolyte layer, the drug layer, the second electrolyte layer, or third electrolyte layer may be covered in its entirety by the first, second, third, or fourth ion exchange layer, respectively. This may prevent formation of a liquid path that passes from the first to fourth ion exchange membranes between the drug layer and the first electrolyte layer, between the second electrolyte layer and the third electrolyte layer, or between the skin and each of these layers, which may degrade the transport number or administration of a drug, or which may reduce stability.

In at least one embodiment, the first conductive layer and/or the second conductive layer may comprise a coating film of a conductive coating. This may further facilitate production process automation or the increase production scale. Furthermore, by using a conductive coating containing a non-metallic conductive filler as the conductive coating, such as carbon powder or carbon fibers, it may be possible to reduce or eliminate the transfer of metallic ions eluted from the first conductive layer and/or the second conductive layer to the living body.

In at least one embodiment, a single substrate may include the first substrate as part thereof and the second substrate as other part thereof, thus further enhancing production process automation or improving the production scale.

In at least one embodiment, a first terminal conductor may be formed on a reverse surface of the first substrate, and the first conductor and the first terminal conductor may be electrically connected to each other via a through-hole that passes through the first substrate. Alternatively, a second terminal conductor may be formed on a reverse surface of the second substrate, and the second conductor and the second terminal conductor may be electrically connected to each other via a through-hole that passes through the second substrate. This may make connection between the iontophoresis device and the power source easier.

In at least one embodiment, a thin battery may be mounted on the surface or the reverse surface of the first substrate and/or the second substrate. This may simplify production, may enhance production process automation, and may increase production scale.

Use of the term “drug” herein refers to a material that has a predetermined medical function or pharmacological function irrespective of the presence or absence of preparation, and is applicable to a living body (a human being or an animal) for the purpose of diagnosis, treatment, recovery, or prevention of disease, promotion or maintenance of health, or the like.

Furthermore, the term “drug ions” as used herein refers to ions that are generated by ionic dissociation of a drug and that have a medical or pharmacological function. The ionic dissociation of a drug may occur by dissolving the drug in a solvent such as water, alcohol, acid, or alkali. The dissociation may also occur by application of a voltage, addition of an ionizing agent, or the like.

The term “first polarity” as used herein refers to electric polarity (positive or negative), and the term “second polarity” refers to an electric polarity (negative or positive) that is opposite to the first polarity.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn are not intended to convey any information regarding the actual shape of the particular elements, and have been solely selected for ease of recognition in the drawings.

FIGS. 1A to 1C are views illustrating a method of producing an active electrode structure according to an embodiment;

FIGS. 2A to 2C are views illustrating a method of producing an active electrode structure according to an embodiment;

FIGS. 3A to 3C are views illustrating a method of producing an active electrode structure according to an embodiment;

FIGS. 4A to 4E are views illustrating a method of producing an active electrode structure according to an embodiment;

FIGS. 5A to 5C are views illustrating a method of producing a counter electrode structure according to an embodiment;

FIGS. 6A to 6E are views illustrating a method of producing a counter electrode structure according to an embodiment;

FIGS. 7A and 7B are views illustrating use forms of an iontophoresis device according to an embodiment;

FIGS. 8A to 8H are views illustrating a method of producing an iontophoresis device according to an embodiment;

FIGS. 9A and 9B are views illustrating use forms of an iontophoresis device according to an embodiment;

FIGS. 10A and 10B are views each illustrating a configuration of a conventional iontophoresis device; and

FIGS. 11A and 11B are views each illustrating a configuration of a conventional iontophoresis device.

### DETAILED DESCRIPTION OF THE INVENTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with iontophoresis devices, controllers, voltage or current sources and/or membranes have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Further more, the particular features, structures, or characteristics may be combined in any suitable manner in one or more embodiments.

The headings provided herein are for convenience only and do not interpret the scope or meaning of the embodiments.

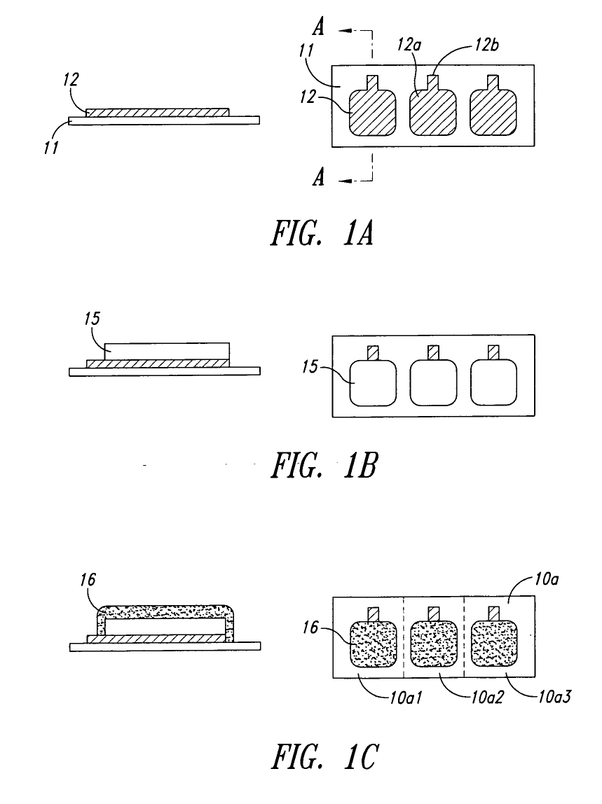

FIGS. 1A to 1C are views showing an example of a method of producing an active electrode structure 10a provided in an iontophoresis device. Plan views in the respective arts are shown on the right side in FIGS. 1A to 6E, and cross sectional views thereof (cross sectional views of a site taken along the line A-A in each of FIGS. 1A, 2A, 3A, 4A, 5A, and 6A) are shown on the left side. The cross sectional views are shown enlarged to a certain degree compared with the plan views. The views are not drawn to scale.

Referring to FIG. 1A, three first conductive layers 12 are formed on an arbitrary insulating substrate 11 such as a phenol board or a glass epoxy board, preferably a flexible substrate 11 made of polyimide, polyester, or the like, comprising three electrode portions 12a with a substantially rectangular shape of about 20 to 50 mm per side, for example, and terminal portions 12b extending from the respective electrode portions 12a.

The first conductive layers 12 may be formed by subjecting a copper-clad board such as FPC to pattern etching, or coating the substrate 11 with a conductive coating. It is preferable that the first conductive layers 12 be formed by applying a conductive coating mixed with a non-metallic conductive filler such as a carbon coating. This may eliminate the possibility that metal eluted from the first conductive layers 12 moves to the living body in the course of administration of a drug.

A drug layer 15 is thus formed by coating the first conductive layers 12 with a drug coating (FIG. 1B).

The drug coating used herein is a coating containing a drug (including a precursor of a drug) whose active ingredient dissociates into positive or negative ions (drug ions) by, for example, being dissolved in a solvent such as water. Examples of the drug whose active ingredient dissociates into positive ions include lidocaine hydrochloride that is an anesthetic and morphine hydrochloride that is an anesthetic. Examples of the drug whose active ingredient dissociates into negative ions include ascorbic acid that is vitamin.

A hydrophilic polymer such as polyvinyl alcohol, polyacrylic acid, polyacrylamide, or polyethylene glycol may be mixed into the drug coating in order to enhance the coating property or film formation property. An appropriate amount a solvent such as water, ethanol, or propanol may be added in order to adjust the viscosity of the drug coating.

It is also possible to add an additional component such as a thickener, a thixo agent, a plasticizer, a defoaming agent, a pigment, an aromatic, a colorant, or a drug stabilizer appropriately in the drug coating.

The drug coating may be applied using an arbitrary process such as screen printing or bar coating using a mask member. The drug coating may be applied so that the entire electrode portion 12a or a part thereof is covered. Alternatively, as shown in FIG. 1B, the drug layer 15 may overlap the electrode portion 12a so that the shape and size of the drug layer 15 become similar to those of the electrode portion 12a.

A first ion exchange layer 16 is thus formed by coating the drug layer 15 with a first ion exchange coating (FIG. 1C).

The first ion exchange coating used herein may contain an ion exchange resin containing an ion exchange group having a counter ion of the same polarity as the drug ions in the drug layer 15. A cation exchange resin may be mixed in the first ion exchange coating when a drug whose active ingredient dissociates into positive drug ions is used in the drug layer 15. An anion exchange resin is mixed in the first ion exchange coating when a drug whose active ingredient dissociates into negative drug ions is used in the drug layer 15, Ion exchange resin may be used as the above mentioned cation exchange resin without any specific limitations placed thereon. A cation exchange group (exchange group whose counter ions are cations) such as a sulfonic group, a carboxylic acid group, or a phosphonic acid group may be introduced into a polymer having a three dimensional network structure such as hydrocarbon resin (e.g., polystyrene resin, acrylic acid resin) or fluorine resin having a perfluorocarbon skeleton. Ion exchange resin may be used as the anion exchange resin without any specific limitations placed thereon. An anion exchange group (exchange group whose counter ions are anions) such as any one of primary to tertiary amino groups, a quaternary ammonium group, a pyridyl group, an imidazole group, a quaternary pyridinium group, or a quaternary imidazolium group may be introduced to a polymer having a three-dimensional network structure similar to the above.

A binder polymer may be mixed into the first ion exchange coating. Thermosetting resin such as phenol resin or methyl methacrylate may be used as the binder polymer, which is capable of maintaining the appropriate dispersed state of ion exchange resin in the first ion exchange coating in the course of the application thereof, and providing the coated first ion exchange layer 16 with insoluble properties with respect to a solvent used in the drug layer 15. Preferably, UV-curable resin of an acrylate, a urethane acrylate, or an epoxy acrylate, capable of providing insoluble property without involving heat treatment may be used. Low molecular-weight polyethylene (paraffin) with a molecular weight of about 10,000 to 40,000, an ultra-high molecular-weight PVA with a molecular weight of 500,000 or more, or chitosan with a molecular weight of about 80,000 insolubilized at pH 7.5 to 9.5 may be used to enhance safety.

In order to enhance the coating properties or the film formation properties, a solvent such as water, ethanol, or propanol may be appropriately mixed with the first ion exchange coating. If an ultra-high molecular weight PVA is used as the binder polymer, it is preferable that the amount of a solvent be reduced to some degree compared with the case of using any other binder polymer. Appropriate components such as a thickener, a thixo agent, a plasticizer, a defoaming agent, a surfactant, a pigment, an aromatic, and a colorant may also be mixed into the first ion exchange coating.

The first ion exchange coating may be applied by an arbitrary procedure such as screen printing or bar coating using a mask member. The first ion exchange layer 16 may be formed so as to partially cover the drug layer 15. However, it is preferable that, as shown in FIG. 1C, the first ion exchange layer 16 be formed so as to cover the entire drug layer 15. It is thus not necessary to provide additional means for preventing a liquid path that passes through the first ion exchange layer 16 from being formed between the drug layer 15 and the skin.

Depending upon the amount of solvent used and the viscosity of the drug coating and the first ion exchange coating, the drug coating and the first ion exchange coating may be mixed with each other during the coating of the first ion exchange layer 16. The amount of solvent and/or viscosity of the drug coating and/or the first ion exchange coating therefore may be adjusted so that the thickness of the drug layer 15 or the first ion exchange layer 16 does not become substantially zero due to mixing. Alternatively, after the drug layer 15 is dried substantially, or at least the surface portion of the drug layer 15 is dried, the first ion exchange coating may be applied thereto.

The first ion exchange layer 16, after being formed, may be treated as a coating film that is insoluble in a solvent such as water. For example, the first ion exchange layer 16 may be irradiated with UV-rays when a UV-curable resin is used as a polymer binder. The first ion exchange layer 16 may be dried or heat-treated at 60° C. to 80° C., when an ultra-high molecular-weight PVA is used. An insoluble coating film can thus be obtained. When a low molecular-weight polyethylene (paraffin) with a molecular weight of about 10,000 to 40,000 is used as a polymer binder, the first ion exchange coating may be applied while heated to about 100° C. to be liquefied during coating, and may be polymerized using irradiation after coating and cooling. An insoluble coating film may thus be obtained. If chitosan, having a molecular weight of about 80,000 and adjusted to pH 7.5 to 9.5, is used as a polymer binder, a coating film insoluble in a solvent such as water may be obtained even without a special treatment after coating provided that the moisture amount during coating is set to be somewhat low.

The first ion exchange layer 16 becomes a semi-permeable film that selectively passes molecules having a size equal to or less than a given size because macropores and micropores are formed in a polymer binder. However, ion exchange resin containing an ion exchange group having a counter ion of the first polarity may be dispersed in the first ion exchange layer 16. Therefore, the first ion exchange layer 16 may function as an ion exchange membrane (ion exchange membrane of a first polarity) that blocks or limits the passage of ions of a second polarity while selectively passing the ions of the first polarity.

An active electrode structure 10a is completed (in the example shown in the figure, three active electrode structures 10a1 to 10a3 are completed) by cutting the substrate 11 along the broken lines shown in FIG. 1C.

The amount of a solvent in the drug layer 15 suitable for administering a drug varies depending upon the type of drug to be administered, and the administration conditions (environment temperature, administration site, etc.) The amount of solvent may not match with the amount used during the coating of the drug layer 15 or the amount present after making the first ion exchange layer 16 insoluble. The amount of solvent in the drug layer 15 may therefore be adjusted by water immersion, drying, or the like after production of the active electrode structure 10a, or prior to the administration of a drug.

The shapes of the conductive layer 12, the drug layer 15, and the first ion exchange layer 16 may be arbitrarily determined. Shapes such as a circle, an oval, or a star may be used instead of the rectangular pattern as shown in the figure.

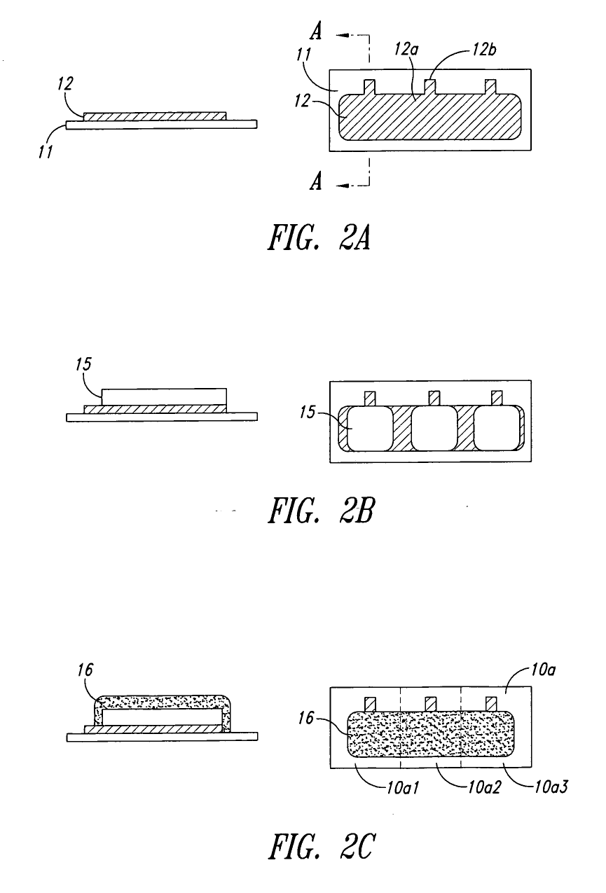

Furthermore, the conductive layer 12 and the ion exchange layer 16 need not be formed as patterns separated on the active electrode structure 10a base as shown in FIGS. 1A to 1C. Referring to FIGS. 2A to 2C, the first conductive layer 12 and/or the first ion exchange layer 16 may also be formed as a continuous pattern. The active electrode structures 10a (10a1 to 10a3) can be produced having the same layer configuration as those of FIGS. 1A to 1C by cutting at the broken lines shown in Figure. Similarly, the drug layer 15 may also be formed as a continuous pattern. In this case, after the drug layer 15 is separated into individual active electrode structures (10a1 to 10a3), it may necessary to add means for preventing a liquid path forming from the drug layer 15 exposed at the cross sectional surface (the severed surface) to the skin.

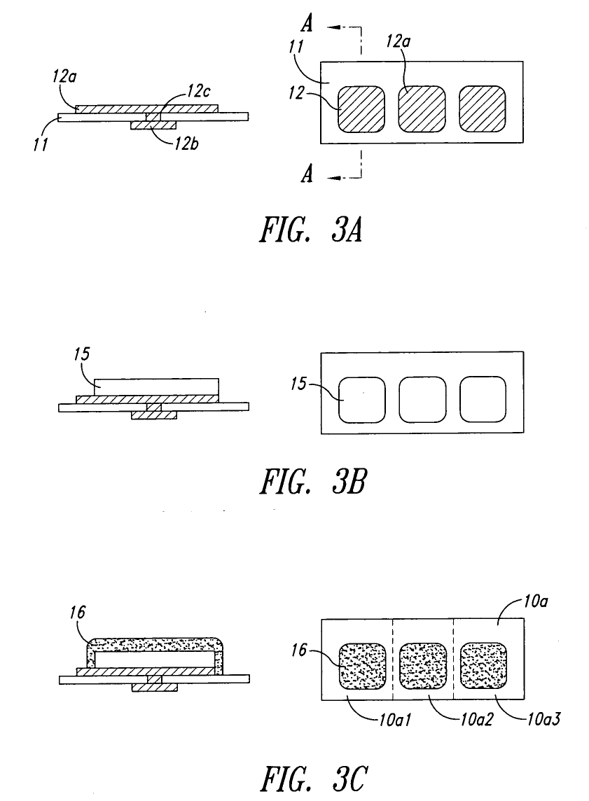

Referring to FIGS. 3A to 3C, making a connection between the substrate 11 and the power source 30 may be made easier by using a substrate having an electrode portion 12a formed on one surface thereof and the terminal portion 12b formed on another surface thereof as the substrate 11. The electrode portion 12a and the terminal portion 12b may be connected via a metal plating such as copper provided in a via or through hole 12c that passes through the substrate 11 or a conductive coating embedded in the through hole 12c.

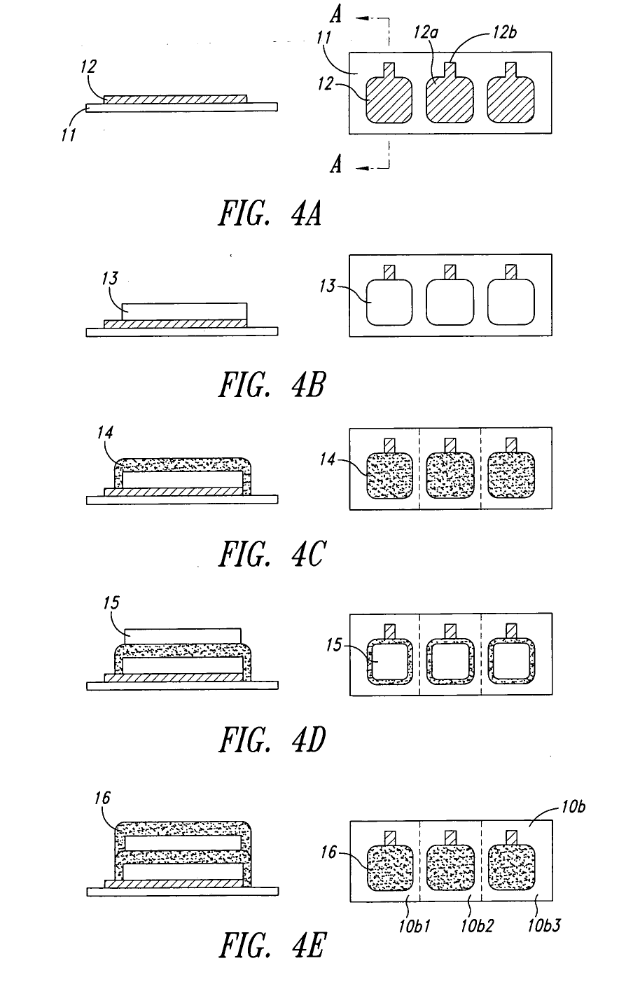

FIGS. 4A to 4C are views showing an example of a method of producing an active electrode structure 10b provided in an iontophoresis device according to another embodiment.

The substrate 11 and the first conductive layer 12 shown in FIG. 4A have the same configurations as those of the substrate 11 and the first conductive layer 12 in the active electrode structure 10a, and may be formed by using methods similar to those used for the active electrode structure 10a.

First electrolyte layers 13 may be formed (FIG. 4B) by coating the first conductive layers 12 with an electrolyte coating.

The electrolyte coating used herein may contain an electrolyte such as NaCl or KCl. It may be preferable to use as the electrolyte an electrolyte that is more likely to be oxidized or reduced before any electrolytic reaction of water occurs (oxidation at a positive electrode and reduction at a negative electrode). For example, an inorganic compound such as ferrous sulfate or ferric sulfate, a medical agent such as ascorbic acid (vitamin C) or sodium ascorbate, an organic acid such as lactic acid, oxalic acid, malic acid, succinic acid, or fumaric acid and/or a salt thereof may be used, thereby helping to suppress the generation of oxygen gas or hydrogen gas. Furthermore, it may also be possible to minimize variations in pH during the passage of current by mixing together a plurality of electrolyte types to become a buffer electrolyte solution when dissolved in a solvent.

A hydrophilic polymer such as PVA, polyacrylic acid, polyacrylamide, or polyethylene glycol may be mixed into the electrolyte coating in order to enhance the coating properties or film formation properties thereof. An appropriate amount of a solvent such as water, ethanol, or propanol may be mixed into the electrolyte coating in order to adjust viscosity.

It is also possible to mix an additional component such as a thickener, a thixo agent, a plasticizer, a defoaming agent, a pigment, an aromatic, or a colorant appropriately into the electrolyte coating.

The electrolyte coating may be applied by using a variety of procedures, such as screen printing or bar coating using a mask member. The electrolyte coating may be applied so that the entire electrode portion 12a or a portion thereof is covered. Alternatively, as shown in FIG. 4B, the first electrolyte layer 13 may overlap the electrode portion 12a so that the shape and size of the first electrolyte layer 13 become the same as those of the electrode portion 12a.

Second ion exchange layers 14 may then be formed by coating the first conductive layers 13 with a second ion exchange coating (FIG. 4C).

The second ion exchange coating used herein contains an ion exchange resin containing an ion exchange group having a counter ion of the opposite polarity to the drug ions in the drug layer 15. An anion exchange resin is mixed in the second ion exchange coating if a drug whose active ingredient dissociates into positive drug ions is used in the drug layer 15. A cation exchange resin is mixed in the second ion exchange coating if a drug whose active ingredient dissociates into negative drug ions is used in the drug layer 15.

Resins similar to those described with respect to the first ion exchange coating may be used as the anion exchange resin and the cation exchange resin for the second ion exchange coating.

A binder polymer may be mixed in the second ion exchange coating. A polymer similar to those described with respect to the first ion exchange coating may be used as the binder polymer.

An appropriate amount of a solvent such as water, ethanol, or propanol may be mixed into the second ion exchange coating in order to enhance the coating properties or film formation properties. In addition, an additional component such as a thickener, a thixo agent, a plasticizer, a surfactant, a pigment, an aromatic, or a colorant may be suitably mixed in.

The second ion exchange coating may be applied by using a variety of procedures, such as screen printing or bar coating using a mask member. The second ion exchange layer 14 may partially cover the first electrolyte layer 13. As shown in FIG. 4C, it may be preferable that the second ion exchange layer 14 cover the entire first electrolyte layer 13. It therefore is not necessary to provide additional means for preventing a liquid path from forming between the first electrolyte layer 13 and the drug layer 15, or between the first electrolyte layer 13 and the skin.

Depending upon the amount of solvent used and the viscosity of the drug coating and the first ion exchange coating, the drug coating and the first ion exchange coating may be mixed with each other during the coating of the second ion exchange layer 14. The amount of solvent and/or viscosity of the drug coating and/or the first ion exchange coating therefore may be adjusted so that the thickness of the first electrolyte layer 13 or the second ion exchange layer 14 does not become substantially zero due to mixing. Alternatively, after the first electrolyte layer 13 is dried substantially, or at least the surface portion of the first electrolyte layer 13 is dried, the second ion exchange coating may be applied thereto.

A process similar to that described for the first ion exchange layer 16 may be performed on the second ion exchange layer 14 in order to make the second ion exchange layer 14 insoluble in a solvent such as water. The second ion exchange layer 14 functions as an ion exchange membrane (ion exchange membrane of the second polarity) that blocks or suppresses the passage of the ions of the first polarity while selectively passing ions of the second polarity by a mechanism similar to that described for the first ion exchange layer 16.

Subsequently, a drug coating and a first ion exchange coating similar to those described above may be successively applied to the second ion exchange layer 14, thus forming the drug layer 15 and the first ion exchange layer 16 are formed (FIGS. 4D and 4E).

The drug coating may be applied by using a variety of procedures, such as screen printing or bar coating using a mask member. The drug layer 15 may be formed to partially overlap with the electrode portion 12a. As shown in FIG. 4D, it may be preferable that the shape and size of the drug layer 15 are set to be similar to those of the electrode portion 12a, and that both are formed so as to substantially overlap. This can maximize the useful area where an ion stream from the drug layer 15 to the electrode portion 12a is generated, while reducing the area of the first ion exchange layer 16 needed to substantially cover the drug layer 15, and hence the amount of the first ion exchange coating material used.

The first ion exchange coating may be applied by using a variety of procedures, such as screen printing or bar coating using a mask member. The first ion exchange layer 16 may be formed to partially cover the drug layer 15. As shown in FIG. 4E, it may be preferable that the first ion exchange layer 16 be formed so as to substantially cover the entire drug layer 15. It thus becomes unnecessary to provide additional means for preventing a liquid path forming between the drug layer 15 and the skin, through the ion exchange layer 16.

The first ion exchange layer 16 may be made insoluble in solvents such as water by using a method similar to that described with respect to the active electrode structure 10a. The first ion exchange layer 16 functions as an ion exchange membrane of the first polarity in a manner similar to that described above.

An active electrode structure 10b may then be completed (in the example shown in the figure, three active electrode structures (10b1 to 10b3) are completed) cutting the substrate 11 at the broken lines shown in FIG. 4E.

For reasons similar to those described above with respect to the active electrode structure 10a, the amount(s) of a solvent in the drug layer 15 and/or the first electrolyte layer 13 may be adjusted by water immersion, drying, or the like after the first and/or the second ion exchange layer(s) 14, 16 are made insoluble, or prior to the administration of a drug.

The first conductive layer 12, the second ion exchange layer 14, and the first ion exchange layer 16 may be formed as a continuous pattern among a plurality of active electrode structures 10b, in a manner similar to that in the active electrode structure 10a. The first conductive layer 12 may comprise the electrode portion 12a formed on one surface of the substrate 11 and the terminal portion 12b formed on the other surface of the substrate 11, and both may be brought into conduction by a via or through hole. Making a connection between the terminal portion 12b and the power source 30 may thus also be enhanced.

The processing of making the ion exchange layers 14 and 16 insoluble in a solvent may be performed each time an ion exchange layer has been formed, or may be performed once after both ion exchange layers have been formed.

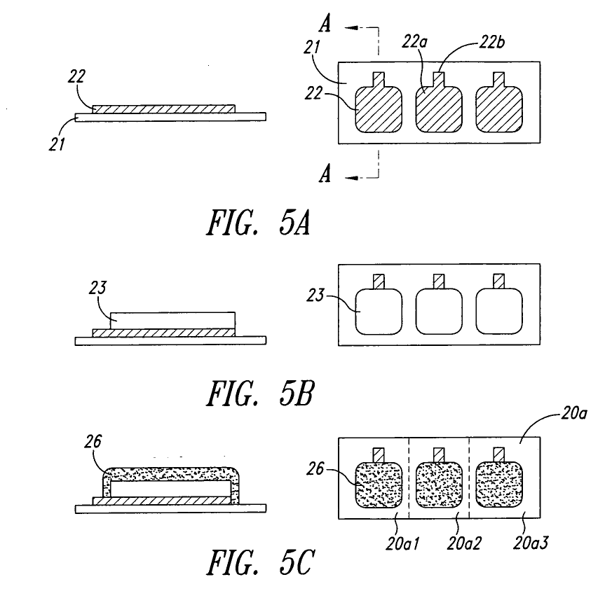

FIGS. 5A to 5C are views showing an example of a method of producing a counter electrode structure 20a.

Referring to FIG. 5A, on a substrate 21, three second conductive layers 22 are formed comprising three electrode portions 22a and terminal portions 22b respectively extending from the electrode portions 22a. The substrate 21 and the second conductive layers 22 may have the configurations similar to those of the substrate 11 and the first conductive layers 12 in the active electrode structure 10a, and may be formed by methods similar to those described for the active electrode structure 10a.

Second electrolyte layers 23 may then be formed (FIG. 5B) by coating the second conductive layers 22 with the same electrolyte coating as that described above, The second electrolyte layers 23 may be formed in a manner similar to that used for the first electrolyte layers 13 of the active electrode structure 10b.

Fourth ion exchange layers 26 may then be formed (FIG. 5C), by coating the second electrolyte layers 23 with the same second ion exchange coating as that described above. The formation of the fourth ion exchange layers 26 and the processing of making them insoluble in a solvent may be performed in a manner similar to that used for the first ion exchange layer 16 of the active electrode structure 10a.

Finally, a counter electrode structure 20b may be completed by cutting the substrate 21 at the broken lines shown in FIG. 5C (in the example shown in the figure, three counter electrode structures (20a1 to 20a3) are completed).

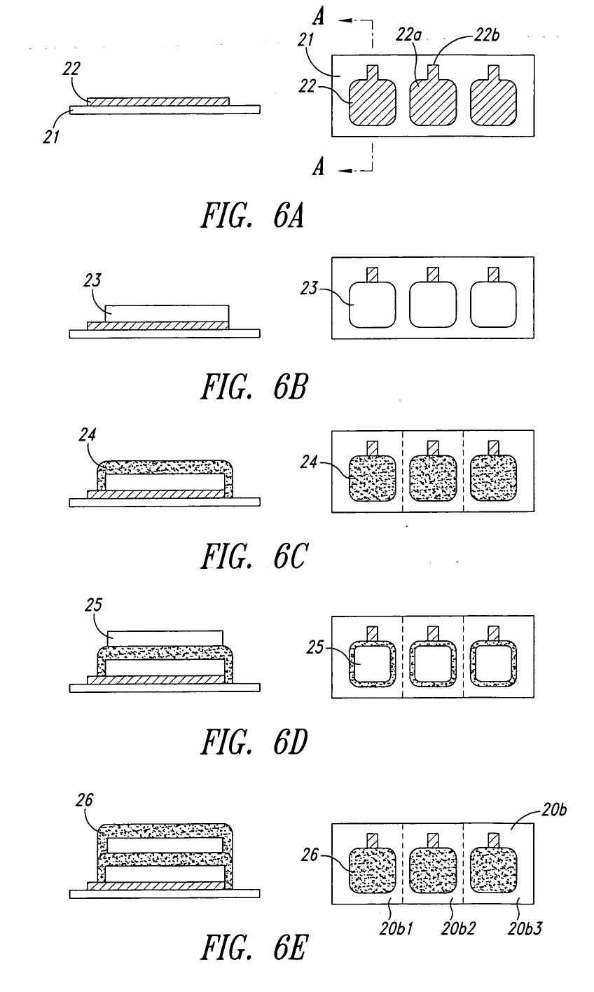

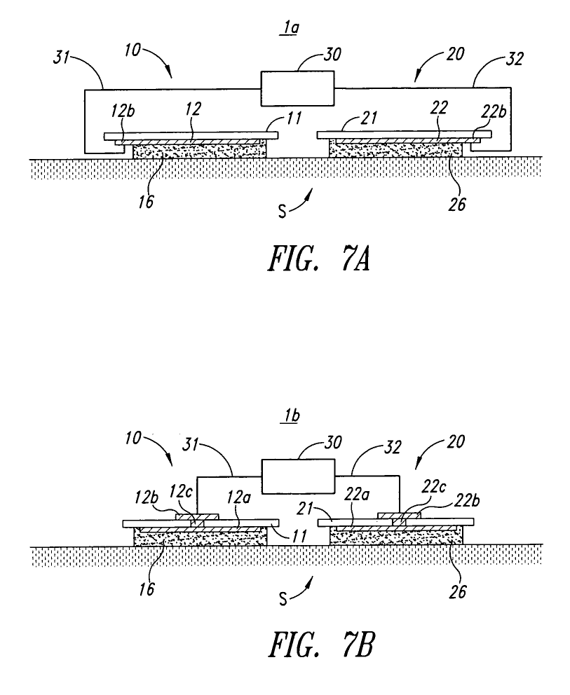

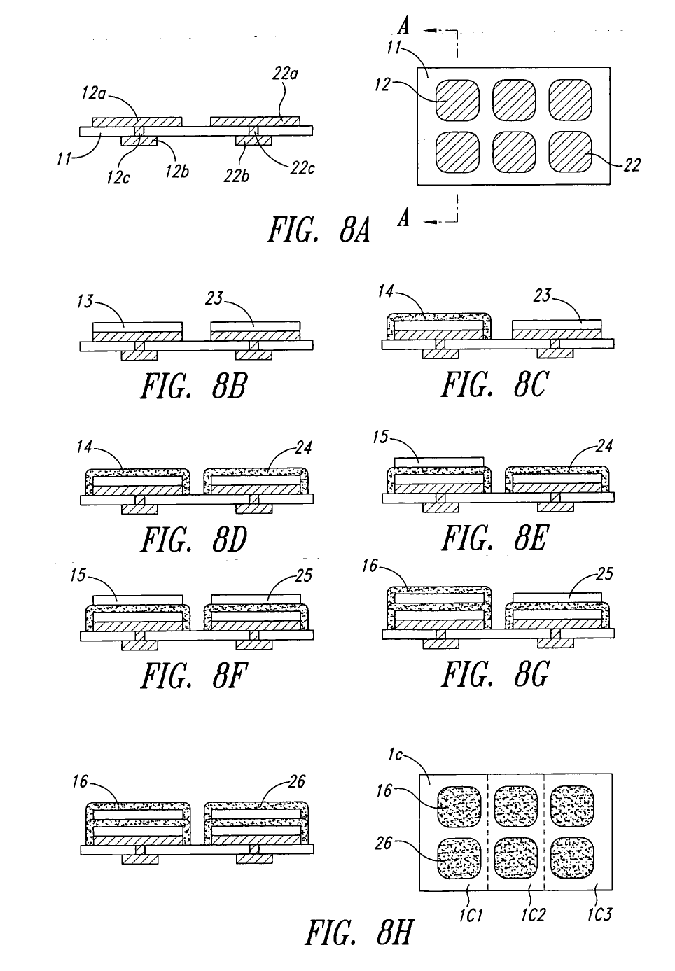

FIGS. 6A to 6E are views showing an example of a method of producing a counter electrode structure.

The substrate 21, the second conductive layers 22, and the second electrolyte layers 23 shown in FIGS. 6A and 6B have the same configurations as those of the substrate 21, the second conductive layers 22, and the second electrolyte layers 23 in the counter electrode structure 20a, and may be formed using the same materials and by the same methods as those described above for the counter electrode structure 20a.

Third ion exchange layers 24 may then be formed (FIG. 6C) by coating the third electrolyte layers 23 using a coating similar to the first ion exchange coating described above. The formation of the third ion exchange layers 24 and processing to make the layers insoluble in a solvent may be performed in a manner similar to those used for the second ion exchange layer 14 of the active electrode structure 10b. The third ion exchange layers 24 function as ion exchange membranes of the first polarity.

Third electrolyte layers 25 may then be formed (FIG. 6D) by coating the third ion exchange layers 24 with a similar electrolyte coating as that described above. The third electrolyte layers 25 may be formed having a similar shape as that of the drug layer 15 of the active electrode structure 10b.

A fourth ion exchange layer 26 may then be formed (FIG. 6E) by coating the third electrolyte layer 25 using a second ion exchange coating similar to that described above. The formation of the fourth ion exchange layer 26 and the processing of making the fourth ion exchange layer insoluble in a solvent such as water may be performed in a manner similar to that used for the first ion exchange layer 16 of the active electrode structure 10b. The fourth ion exchange layer 26 functions as an ion exchange membrane of the second polarity in the same way as described above.

Finally, a counter electrode structure 20b may be completed (in the example shown in the figure, three counter electrode structures (20b1 to 20b3) are completed) by cutting the substrate 21 at the broken lines shown in FIG. 6E.

The second conductive layers 22, the third ion exchange layers 24, and the fourth ion exchange layers 26 may be formed as a continuous pattern among a plurality of counter electrode structures 20b when producing the counter electrode structures 20a and 20b. The second conductive layer 22 may comprise an electrode portion 22a formed on one surface of the substrate 21 and a terminal portion 22b formed on the other surface of the substrate 21, and both may be connected by a via or through hole, thus making it easier to form a connection between the terminal portion 22b and the power source 30.

The processing of making the third and fourth ion exchange layers 24 and 26 insoluble in a solvent may be performed each time an ion exchange layer has been formed, or may be performed once after both the ion exchange layers have been formed.

The amount of solvent in the second and/or third electrolyte layer(s) 23 and/or 25 may be adjusted by water immersion, drying, or the like after the of the third and/or the fourth ion exchange layer(s) 24 and/or 26 are made insoluble, or prior to the administration of a drug, for reasons similar to those described with respect to the active electrode structure 10a.

FIGS. 7A and 7B are views that illustrate forms of iontophoresis devices 1a and 1b each having the active electrode structure 10 (10a or 10b) and the counter electrode structure 20 (20a or 20b) produced as described above. In FIGS. 7A and 7B, the first to third electrolyte layers 13, 23, and 25, the drug layer 15, and the second and third ion exchange layers 14 and 24 have been omitted. Only the substrates 11 and 21, the first conductive layers 12 and 22, and the first and fourth ion exchange layers 16 and 26 are shown.

Connecting leads 31 and 32 from the power source 30 to the terminal portions 12b and 22b in the iontophoresis device 1a shown in FIG. 7A allow current to flow under the condition that the first and the fourth ion exchange layers 16 and 26 are kept in electrical contact with skin S of a living body. Drug ions in the drug layer 15 are thus administered to the skin S via the first ion exchange layer 16.

The leads 31 and 32 and the terminal portions 12b and 22b may be connected to each other by using any of a variety of structures and/or methods. For example, solder, a conductive adhesive, a conductive adhesive tape, or the like may be used. Connection may also be facilitated by attaching connectors to the terminal portions 12b and 22b and the leads 31 and 32.

A positive electrode of the power source 30 is connected to the terminal portion 12b of the active electrode structure 10, and a negative electrode thereof is connected to the terminal portion 22b of the counter electrode structure 20 when a drug whose active ingredient dissociates into positive drug ions is mixed into the drug layer 15. The negative electrode of the power source 30 is connected to the terminal portion 12b of the active electrode structure 10, and the positive electrode is connected to the terminal portion 22b of the counter electrode structure 20 when a drug whose active ingredient dissociates into negative drug ions is mixed into the drug layer 15.

The transport number and administration efficiency of a drug may be increased, and the safety and stability of the administration of a drug may be enhanced in a manner similar to that the mentioned iontophoresis devices 101a and 101b described above when any of the active electrode structures 10a and 10b is used as the active electrode structure 10, or when any of the counter electrode structures 20a and 20b is used as the counter electrode structure 20.

Furthermore, the first to third electrolyte layers 13, 23, and 25, the drug layer 15, and the first to fourth ion exchange layers 14, 16, 24, and 26 may be formed by using a coating method. Therefore, it is possible to employ automated production processes or increase the production scale by using multiple patterning, for example. In addition, potential problems related to wasted material due to cutting margins or punching margins when forming the ion exchange membrane may be resolved.

Completely covering the drug layer 15 and the first to third electrolyte layers 13, 23, and 25 by using the first to fourth ion exchange layers 14, 16, 24, and 26, respectively, immediately above the drug layer 15 may help to prevent the following problem: formation of a liquid path between the first electrolyte layer 13 and the drug layer 15, between the second electrolyte layer 23 and the third electrolyte layer 25, or between each of these layers and the skin S, the path passing through the ion exchange layers 14, 16, 24, or 26, which may result in the transport number, the administration efficiency of a drug, safety, and/or stability being reduced.

FIG. 7B shows a form of the iontophoresis device 1b that uses the active electrode structure 10 (10a or 10b) in which the electrode portion 12a is formed on one surface of the substrate 11, the terminal portion 12b is formed on the other surface of the substrate 11, and the electrode portion 12a and the terminal portion 12b are electrically connected to each other by the via or through hole 12c. The iontophoresis device 1b also uses the counter electrode structure 20 (20a or 20b) in which the electrode portion 22a is formed on one surface of the substrate 21, the terminal portion 22b is formed on the other surface of the substrate 21, and the electrode portion 22a and the terminal portion 22b are electrically connected to each other via the through hole 22c.

Similar functional effects as those described with respect to the iontophoresis device 1a can also be achieved with the iontophoresis device 1b. The leads 31 and 32 and the terminal portions 12b and 22b may be connected to each other in an arbitrary manner in a manner similar to that used for the iontophoresis device 1a. The terminal portions 12b and 22b will be positioned on the opposite side of the skin S with the iontophoresis device 1b. Additional functional effects such as easy connection operation of the leads 31 and 32 and insulation between the connection sites and the skin S may thus be achieved.

FIGS. 8A to 8H are views that each illustrate a production process of an iontophoresis device 1c. FIGS. 8A and 8H show both a cross sectional views and plan views, while FIGS. 8B to 8G show only cross sectional views.

Referring to FIG. 8A, first conductive layers 12 and second conductive layers 22 are formed on a substrate 11. Electrode portions 12a and 22a formed on one surface of the substrate 11, and terminal portions 12b and 22b formed on another surface of the substrate 11 are connected together via through holes 12c and 22c that pass through the substrate 11.

The substrate 11 may be formed by using a method that is similar to the method described for the active electrode structure 10a.

First and second electrolyte layers 13 and 23 may then be formed (FIG. 8B) by coating the first and second conductive layers 12 and 22 with an electrolyte coating similar to that described above.

A second ion exchange layer 14 may then be formed (FIG. 8C) by coating the first electrolyte layer 13 with a similar second ion exchange coating as that described above. A third ion exchange layer 24 may be formed (FIG. 8D) by coating the second electrolyte layer 23 with a similar first ion exchange coating as that described above. Processing to make the ion exchange layers 14 and 24 insoluble in a solvent such as water is then performed.

A drug layer 15 may then be formed (FIG. 8E) by coating the second ion exchange layer 14 with a similar drug coating as that described above. A third electrolyte layer 25 may be formed (FIG. 8F) by coating the third ion exchange layer 24 with a similar electrolyte coating as that described above.

A first ion exchange layer 16 may then be formed (FIG. 8G) by coating the drug layer 15 with a similar first ion exchange coating as that described above, and a fourth ion exchange layer 26 may be formed (FIG. 8H) by coating the third electrolyte layer 25 with a similar second ion exchange coating as that described above. Processing to make the respective ion exchange layers 16 and 26 insoluble in a solvent such as water may then be performed.

The iontophoresis device 1c (in the example shown in the figure, three ionphotoresis devices (1c1, 1c2, 1c3)) may then be completed by cutting the substrate 11 at the broken lines shown in FIG. 8H.

The drug layer 15, the first to third electrolyte layers 13, 23 and 25, and the first to fourth ion exchange layers 14, 16, 24, and 26 may be formed by using the methods similar to those employed when forming the corresponding layers of the active electrode structures 10a and 10b and the counter electrode structures 20a and 20b.

The amount of a solvent in at least one of the drug layer 15, and the first, second, and third electrolyte layers 13, 23, and 25 may be adjusted by water immersion, drying, or the like after at least one of the first, second, third, and fourth ion exchange layers 14, 16, 24, and 26 has been made insoluble, or prior to the administration of a drug, for reasons similar to those described above for the active electrode structure 10a.

The acts (a) through (h) need not be performed in the order described above. For example, the iontophoresis device 1c may also be produced by performing processes in the following order: FIG. 8A→FIG. 8B→FIG. 8C→FIG. 8E→FIG. 8G→FIG. 8D→FIG. 8F→FIG. 8H.

Processing to make the ion exchange layers 14, 16, 25, and 26 insoluble in a solvent may be performed each time each ion exchange layer has been formed, or may be performed once two or more ion exchange layers have been formed.

Formation of the first and second conductive layer 12 and 22, and formation of the first and third electrolyte layers 13 and 25 may be performed in one act. An advantage thus exists in that the number of production steps may be reduced compared to the iontophoresis device 1a formed of the active electrode structure 10b and the counter electrode structure 20b.

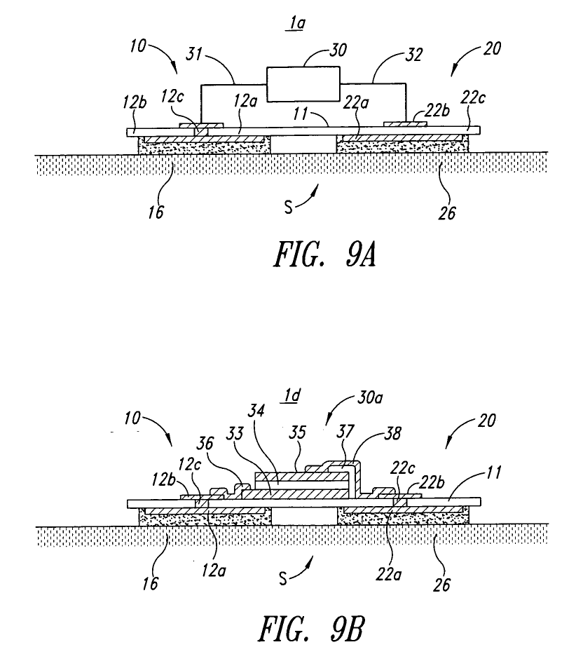

FIG. 9A is a view that illustrates an example of the iontophoresis device 1c, with the first to third electrolyte layers 13, 23, and 25, the drug layer 15, and the second and third ion exchange layers 14 and 24 omitted.

As shown, with the iontophoresis device 1c, current is allowed to pass through the connecting leads 31 and 32 from the power source 30 to the terminal portions 12b and 22b in a manner similar to that of the iontophoresis device 1b provided that the first and third ion exchange layers 16 and 26 are kept in contact with the skin S of a living body. Drug ions in the drug layer 15 are thus administered to the skin S via the first ion exchange layer 16.

FIG. 9B is a view that illustrates the configuration of an iontophoresis device 1d.

The iontophoresis device 1d includes an active electrode structure 10 and a counter electrode structure 20 formed in the manner similar to the iontophoresis device 1c. In addition, a thin battery 30a having a first active electrode layer 33, a separator layer 34, and a second active electrode layer 35 formed by a coating method such as printing are mounted on one surface of the substrate 11. The first active electrode layer 33 and the terminal portion 12b may be connected together via a coating film 36 of a conductive coating, and the second active electrode layer 35 and the terminal portion 22b may be connected together via a coating film 38 of a conductive coating formed via an insulating paste layer 37.

All of the active electrode structure 10, the counter electrode structure 20, and the battery 30a may be formed by employing a coating method In iontophoresis device 1d. Functional effects such as added simplification of producing the iontophoresis device and an additional reductions in production cost may therefore be achieved. Furthermore, the ease and exactness of the connection between the battery 30a and each of the terminal portions 12b and 22b may be further enhanced.

Regarding the above mentioned thin battery 30a, various kinds of configurations and production methods are known, and thin battery having various configurations produced by various methods and chemistries may be used. For example, it is possible to use a thin battery having any of the configurations produced by any one of the production methods disclosed in JP 11-067236 A, U.S. Patent Publication No. 2004/0185667 A1, and U.S. Pat. No. 6,855,441, which are herein incorporated by reference in their entirety to the extent that contradictions with the present disclosure do not exist.

The present invention has been described by way of several embodiments. It should be noted that the present invention is not limited to these embodiments, and may be suitably altered within the scope of the claims.

For example, in the above embodiments, a case has been described where three active electrode structures, counter electrode structures, or iontophoresis devices are formed on one substrate. The number of patterns formed on a single substrate is arbitrary, however. A single active electrode structure, counter electrode structure, or iontophoresis device may be formed on one substrate, or two, four, or more active electrode structures, counter electrode structures, or iontophoresis devices may be formed on one substrate.

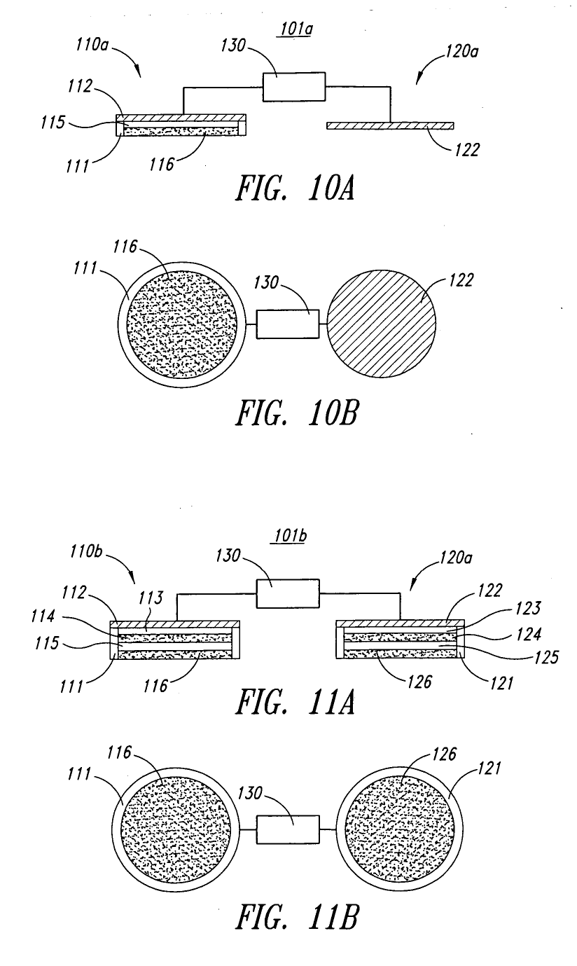

FIGS. 7A and 7B exemplify an iontophoresis device in which the active electrode structure 10a or 10b is combined with the counter electrode structure 20a or 20b. It is also possible to configure the iontophoresis device by combining the active electrode structure 10a or 10b with the counter electrode structure 120a or 120b shown in FIGS. 10A-10B and 11A-11B.

A counter electrode structure need not be provided in the iontophoresis device itself, provided that the active electrode structure 10a or 10b is brought into electrical contact with the skin of a living body, a part of the living body is brought into contact with a ground member to be an earth, and a voltage is applied to the active electrode structure 10a or 10b to administer a drug. This iontophoresis device may be capable of administering a drug at a high transport number and efficiency, and material loss and production cost may be further reduced. Automated production and increases in production scale may also be realized. Thus, the basic functional effects of the present invention may be achieved, such iontophoresis devices are also included in the scope of the present invention.

Further, FIG. 9 shows a case where the thin battery 30a is placed on the opposite side of the contact surface of the substrate 11 with respect to the skin. The thin battery 30a and the terminal portions 12b and 22b may be placed on the same side of the contact surface of the substrate 11 as that of the skin. All coating acts are thus performed only on one surface of the substrate 11, so that production processing may be further simplified. Furthermore, the thin battery 30a may also be used in combination with the iontophoresis device 1a or 1b. The thin battery 30a may be mounted on the front surface or the reverse surface of the substrate 11 or 21. Furthermore, the terminal portions 12b and 22b may be connected to the battery 30a by an arbitrary method such as the use of a lead or a connector instead of using the conductive coating thin films 36 and 38.

Each of the active electrode structures and the counter electrode structures shown herein may also include an additional layer configuration such as a liner for protection from drying, foreign matter, and the like. The iontophoresis device may further include additional members such as a switch for the passage of a current or a control device.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

The above description of illustrated embodiments, including what is described in the Abstract, is not intended to be exhaustive or to limit the invention to the precise forms disclosed. Although specific embodiments of and examples are described herein for illustrative purposes, various equivalent modifications may be made without departing from the spirit and scope of the invention, as will be recognized by those skilled in the relevant art. The teachings provided herein of the invention may be applied to other medical devices, not necessarily the exemplary iontophoresis device generally described above.

What is claimed is:

1. An iontophoresis device for administering drug ions of a first polarity, generated by dissociation of a drug, to a living body, comprising an active electrode structure comprising:
a first conductive layer formed on a surface of a first substrate;
a drug layer comprising a drug coating containing the drug, the drug layer being deposited on the first conductive layer; and
a first ion exchange layer comprising of an ion exchange coating containing an ion exchange resin containing an exchange group having a counter ion to the first polarity ions, the first ion exchange layer being deposited on the drug layer.

2. An iontophoresis device according to claim 1, wherein the active electrode structure further comprises:
a first electrolyte layer comprising an electrolyte coating containing an electrolyte, the first electrolyte layer being deposited on the first conductive layer; and
a second ion exchange layer comprising an ion exchange coating containing an ion exchange resin containing an exchange group having a counter ion to a second polarity ion, the second ion exchange layer being deposited on the first electrolyte layer,
wherein the drug layer is deposited on the second ion exchange layer.

3. An iontophoresis device according to claim 1, further comprising a counter electrode structure comprising:
a second conductive layer formed on a surface of a second substrate;
a second electrolyte layer comprising an electrolyte coating containing an electrolyte, the second electrolyte layer being deposited on the second conductive layer;
a third ion exchange layer comprising an ion exchange coating containing an ion exchange resin containing an exchange group having a counter ion to the first polarity ions, the third ion exchange layer being deposited on the second electrolyte layer;
a third electrolyte layer comprising an electrolyte coating containing an electrolyte, the third electrolyte layer being deposited on the third ion exchange layer; and
a fourth ion exchange layer comprising an ion exchange coating containing an ion exchange resin containing an exchange group having a counter ion to the second polarity ions, the fourth ion exchange layer being deposited on the third electrolyte layer.

4. An iontophoresis device according to claim 1, wherein at least one of the drug coating and the electrolyte coating further contains a water-soluble polymer.

5. An iontophoresis device according to claim 1, wherein at least one of the first ion exchange layer, the second ion exchange layer, the third ion exchange layer, and the fourth ion exchange layer comprises a non-water-soluble coating film.

6. An iontophoresis device according to claim 5, wherein the ion exchange coating further contains one of a low molecular-weight polyethylene, ultra-high molecular weight PVA, chitosan, and a mixture thereof.

7. An iontophoresis device according to claim 1, wherein the drug layer is covered in its entirety by the first ion exchange layer.

8. An iontophoresis device according to claim 2, wherein the first electrolyte layer is covered in its entirety by the second ion exchange layer.

9. An iontophoresis device according to claim 3, wherein the second electrolyte layer is covered in its entirety by the third ion exchange layer.

10. An iontophoresis device according to claim 3, wherein the third electrolyte layer is covered in its entirety by the fourth ion exchange layer.

11. An iontophoresis device according to claim 3, wherein at least one of the first conductive layer and the second conductive layer comprises a coating film of a conductive coating.

12. An iontophoresis device according to claim 11, wherein the conductive coating contains a non-metallic conductive filler.

13. An iontophoresis device according to claim 3, wherein a single substrate has the first substrate as portion thereof and the second substrate as another portion thereof.

14. An iontophoresis device according to claim 3, wherein:
a first terminal conductor is formed on a reverse surface of the first substrate, and
the first conductor layer and the first terminal conductor are electrically connected to each other via a through-hole that passes through the first substrate.

15. An iontophoresis device according to claim 3, wherein:
a second terminal conductor is formed on a reverse surface of the second substrate, and
the second conductor layer and the second terminal conductor are electrically connected to each other via a through-hole that passes through the second substrate.

16. An iontophoresis device according to claim 3, wherein a thin battery is mounted on one of the surface and a reverse surface of at least one of the first substrate and the second substrate.

17. A method of producing an iontophoresis device for administering drug ions of a first polarity generated by dissociation of a drug to a living body, comprising:
forming a first conductive layer on a surface of a first substrate;
forming a drug layer by applying a drug coating containing the drug to the first conductive layer; and
forming a first ion exchange layer by applying an ion exchange coating containing an ion exchange resin containing an exchange group having a counter ion to the first polarity ions to the drug layer.

18. A method of producing an iontophoresis device according to claim 17, further comprising:
forming a first electrolyte layer by applying an electrolyte coating containing an electrolyte to the first conductive layer; and
forming a second ion exchange layer by applying an ion exchange coating containing an ion exchange resin containing an exchange group having a counter ion to second polarity ions to the first electrolyte layer,
wherein the drug layer is formed on the second ion exchange layer.

19. A method of producing an iontophoresis device according to claim 18, wherein at least one of the drug coating and the electrolyte coating further contains a water-soluble polymer.

20. A method of producing an iontophoresis device according to claim 18, further comprising setting at least one of the first ion exchange layer, the second ion exchange layer, the third ion exchange layer, and the fourth ion exchange layer to be non-water-soluble.

21. A method of producing an iontophoresis device according to claim 18, wherein the ion exchange coating further contains one of a low molecular-weight polyethylene, ultra-high molecular-weight PVA, chitosan, and a mixture thereof.

22. A method of producing an iontophoresis device according to claim 18, wherein the drug layer is covered in its entirety by the first ion exchange layer.

23. A method of producing an iontophoresis device according to claim 17, comprising:
forming a second conductive layer to a surface of a second substrate;
forming a second electrolyte layer by applying an electrolyte coating containing an electrolyte to the second conductive layer;
forming a third ion exchange layer by applying an ion exchange coating containing an ion exchange resin containing an exchange group having a counter ion to the first polarity ions to the second electrolyte layer;
forming a third electrolyte layer by applying an electrolyte coating containing an electrolyte to the third ion exchange layer; and
forming a fourth ion exchange layer by applying an ion exchange coating containing an ion exchange resin containing an exchange group having a counter ion to the second polarity ions to the third electrolyte layer.

24. A method of producing an iontophoresis device according to claim 23, wherein at least one of the drug coating and the electrolyte coating further contains a water-soluble polymer.

25. A method of producing an iontophoresis device according to claim 23, further comprising setting at least one of the first ion exchange layer, the second ion exchange layer, the third ion exchange layer, and the fourth ion exchange layer to be non-water-soluble.

26. A method of producing an iontophoresis device according to claim 25, wherein the ion exchange coating further contains one of a low molecular-weight polyethylene, ultra-high molecular-weight PVA, chitosan, and a mixture thereof.

27. A method of producing an iontophoresis device according to claim 23, wherein the drug layer is covered in its entirety by the first ion exchange layer.

28. A method of producing an iontophoresis device according to claim 23, wherein the first electrolyte layer is covered in its entirety by the second ion exchange layer.

29. A method of producing an iontophoresis device according to claim 23, wherein the second electrolyte layer is covered in its entirety by the third ion exchange layer.

30. A method of producing an iontophoresis device according to claim 23, wherein the third electrolyte layer is covered in its entirety by the fourth ion exchange layer.

31. A method of producing an iontophoresis device according to claim 23, wherein at least one of the first conductive layer and the second conductive layer is formed by application of a conductive coating.

32. A method of producing an iontophoresis device according to claim 31, wherein the conductive coating contains a non-metallic conductive filler.

33. A method of producing an iontophoresis device according to claim 23, wherein a single substrate has the first substrate as portion thereof and the second substrate as another portion thereof.

34. A method of producing an iontophoresis device according to claim 23, wherein:
a first terminal conductor is formed on a reverse surface of the first substrate, and
the first conductor layer and the first terminal conductor are electrically connected to each other via a through-hole that passes through the first substrate.

35. A method of producing an iontophoresis device according to claim 23, wherein:
a second terminal conductor is formed on a reverse surface of the second substrate, and
the second conductor layer and the second terminal conductor are electrically connected to each other via a through-hole that passes through the second substrate.

36. A method of producing an iontophoresis device according to claim 23, further comprising mounting a thin battery on one of the surface and a reverse surface of at least one of the first substrate and the second substrate.
